(19) United States
(12) Patent Application Publication
(10) Pub. No.: US 2026/0204369 A1
(43) Pub. Date: Jul. 16, 2026

(54) AMBIENT ARTIFICIAL INTELLIGENCE MODEL THAT TRANSFORMS UNSTRUCTURED DATA INTO STRUCTURED DATA FOR AUTOMATIC POPULATION OF A PROM

(71) Applicant: Christopher West MD, P.C., Murray, UT (US)

(72) Inventor: Christopher Reid WEST, Holladay, UT (US)

(21) Appl. No.: 19/024,175

(22) Filed: Jan. 16, 2025

(51) Int. Cl. G16H 10/60; G06F 40/30; G10L 15/26

(52) U.S. Cl. CPC G16H 10/60; G06F 40/30; G10L 15/26

(57) ABSTRACT

Techniques for transforming unstructured data into structured data to automatically populate a PROM are disclosed. A service detects a keyword or topic included in a transcribed output. Both the keyword and the topic are related to a specific health measure of a patient. The service transitions from operating in a passive observation state to operating in an active observation state. The service uses NLP to apply a semantic meaning to a set of transcribed output stored in a buffer. The service applies a quantitative value, based on the semantic meaning, to the set of transcribed output, thereby transforming the set of transcribed output from being unstructured data to being structured data. The service uses the structured data to automatically populate the PROM. The completed PROM score is then stored in a format that can be stored in a database or manually or automatically uploaded into an EMR.

### BACKGROUND

Significant medical advancements are made each year. As some examples, significant improvements and efforts are made each year in the realm of hip replacement, knee replacement, arthritis management, and many other areas of healthcare. For instance, each year, tens of thousands of hip and knee replacement surgeries are performed, resulting in significant improvements to patients'lifestyles and qualities of life. The medical community is always seeking ways to try to improve the administration of medicine.

Many medical practitioners are now transitioning to value-based care. This type of care enables practitioners and health systems to provide the highest quality of care to patients at the lowest cost. The current system prioritizes, incentivizes, and pays for procedures, effectively paying more when more procedures are performed, regardless of whether patients see improvement in their health along the way. Value-based care represents a revolutionary shift in this paradigm to link payment and compensation structures to improvement of patient health. It is particularly challenging to achieve such a shift without the ability to measure a patient's current health status.

Patient Reported Outcome Measures (PROMs) are an effective tool that has been developed to assess, from the patient's perspective, a measurement of his/her health for a given condition. A PROM is a standardized survey report, filled out by the patient. This report is used to help determine a patient's health and well-being at a given point in time. Historically, PROMs were filled out by the patient with pen and paper and there have been significant developments in the last two decades to improve the patient experience with electronic PROM surveys. Until fairly recently, the medical community has generally not collected Patient Reported Outcome Measures (PROMs) for purposes other than research, and were used mostly in the academic setting. Recently, however, billing entities are requiring the collection of PROMs to help determine the cost efficiency of certain treatments or surgeries.

FIG. 1A shows an example of a patient reported outcome measure PROM 100. Typically, the patient will receive the PROM 100 and will be tasked with completing the PROM 100 at a given point in time, such as before a surgery and/or after a surgery. PROM 100 includes an option for the patient to provide an analog score 105 in which the patient can mark on the line chart a relative level of pain, difficulty, or other metric. This analog score represents a quantitative value indicative of the patient's health measure. Other types of PROMs exist as well, such as PROMs that include discrete selectable options (e.g., select a value from 1-5 or a number of checkbox options), as shown in FIG. 1B. These other types of PROMs can also be used to objectively measure the patient's level of pain or difficulty in completing a task. For instance, FIG. 1B shows a PROM 110 that includes discrete, selectable options (e.g., option 115) for selecting an answer to a given question.

Another type of PROM is called a “Promis” PROM or a “Promis” score. This Promis score is another standardized scoring technique that dynamically modifies subsequent questions based on the responses a patient provided for an earlier question. The Promis score is designed to try to decrease the amount of survey burden placed on a patient. The Promis score uses branching logic to determine which questions to present to a patient in order to evaluate the patient as quickly and as effortlessly as possible.

The Centers for Medicare and Medicaid Services (CMS) is now requiring medical practitioners to submit PROM data to CMS. For example, facilities are now being required to collect and submit PROMs to CMS for joint replacements in order to have their billing requests approved. Currently, CMS is requiring PROM reporting for a minimum of 50% of inpatient joint replacement surgeries, with a plan to expand this requirement to outpatient joint replacement surgeries by the year 2027. These reports must show a certain amount of progressive improvement on the PROMs in order to qualify for full payment from CMS. If the hospital does not collect and provide these PROMs, then CMS is implementing a penalty against the hospital and will potentially reduce the amount of reimbursement the hospital will receive from CMS with a risk of loss of 25% of annual payment update. If a hospital performs several thousand joint replacements over the span of a single year, the liability for that hospital may now be multiple millions of dollars per year if the hospital does not collect and report the needed PROM data.

Perhaps the primary hurdle with the PROM data relates to the collection phase. It has proven to be quite a challenge to have patients self-report using a PROM form. One of the more successful techniques for collecting PROMs has been during a check-in appointment at a clinic. For instance, when a medical practitioner meets with a patient, the medical practitioner can sit with the patient and together they can complete the PROM. This technique has proven to be generally successful with regards to the collection phase. After the data is collected, the data can then be uploaded to a repository, such as a database, for submission to CMS.

Often, medical practitioners see many dozens of patients per day. The amount of time a medical practitioner has with a patient is also limited. Many medical practitioners have found that spending time on administrative procedures (e.g., the completion of a PROM) reduces the amount of time the medical practitioner has to diagnose and treat a patient. Thus, it is often the case that the completion of the PROM is de-prioritized during the in-person meetings.

The need still exists, however, to efficiently acquire PROM data for submission to CMS. What is especially needed, therefore, is a streamlined, efficient, and intuitive manner for medical practitioners and their teams to collect PROM data so as to satisfy the demands of CMS and to help improve the shared decision making process between medical practitioner and patient related to selection of an appropriate treatment course. Collecting PROM data not only helps with billing purposes, but it also helps advance medicine by assisting in evaluating which procedures are effective for a given condition and which procedures can be improved. Collecting PROM data is an indispensable component of eliminating waste to help reduce the cost of medical care over time.

The subject matter claimed herein is not limited to embodiments that solve any disadvantages or that operate only in environments such as those described above. Rather, this background is only provided to illustrate one exemplary technology area where some embodiments described herein may be practiced.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings in which:

FIG. 1A and FIG. 1B illustrate examples of PROMs.

FIG. 2 illustrates an example computing architecture in which a service transforms unstructured data into structured data.

FIG. 3 illustrates an example scenario involving an ambient artificial intelligence (AI) device.

FIG. 4 illustrates an automatically completed PROM that is completed using structured data.

FIG. 5 illustrates an example collection technique for collecting unstructured data that is to be transformed into structured data.

FIG. 6 illustrates an example of additional input data used to generate the structured data.

FIG. 7 illustrates an example of input data.

FIGS. 8, 9, and 10 illustrate various different charts that plot data relating to user scores.

FIG. 11 illustrates a flowchart of an example method for transforming unstructured data into structured data, which is then used to automatically populate a PROM.

FIG. 12 illustrates an example computer system that can be configured to perform any of the disclosed operations.

### DETAILED DESCRIPTION

As mentioned earlier, medical practitioners see many dozens of patients per day. The amount of time a medical practitioner has with a patient is limited. Many medical practitioners have found that spending time on administrative procedures (e.g., the completion of a PROM) reduces the amount of time the medical practitioner has to diagnose and treat a patient. Thus, it is often the case that the completion of the PROM is de-prioritized during the in-person meetings as practitioners often feel that their clinical gestalt is an adequate substitute.

In view of heighted reporting requirements now being placed on medical practitioners, there is a growing need to efficiently acquire PROM data for submission to CMS. What is especially needed is a streamlined, efficient, and intuitive manner for medical practitioners and their teams to collect PROM data so as to satisfy the demands of CMS and to help improve and direct the types of treatments that are provided to patients. Collecting PROM data not only helps with billing purposes, but it also helps advance medicine by assisting in evaluating which treatments are effective, how they can be improved, and which treatments are appropriate for the condition of the patient. Collecting PROM data is also relevant to help reduce the cost of medical care over time.

The disclosed embodiments bring about numerous benefits, advantages, and practical applications to how unstructured data (e.g., user input) is transformed into structured data, which is then used to automatically populate a PROM. By following the disclosed principles, the data needed to complete a PROM is now automatically collected, analyzed, formatted, transformed, and repurposed into a format that is suitable for automatic entry into a PROM. The disclosed operations significantly improve a medical practitioner's efficiency and also satisfy the heightened reporting requirements that are placed on medical practitioners. The disclosed embodiments provide a streamlined, efficient, and intuitive manner for medical practitioners and their teams to process PROM data.

Beneficially, the disclosed embodiments can be implemented as an ambient AI device that listens, observes, or otherwise has access to a patient. The ambient AI device, which can be referred to more generally as a “service,” is tasked with collecting input from the patient (often unstructured input) and transforming the unstructured input into structured data. The format of this structured data is advantageously designed to align with the format of a PROM. Because of this alignment, the structured data can be used to automatically populate the fields of the PROM. While a majority of the examples recited herein are focused on scenarios in which the ambient AI device is “listening” to a conversation between a patient and a medical practitioner, a person skilled in the art will appreciate how other data collection techniques can be employed as well, such as image analysis techniques, video analysis techniques, text analysis techniques, behavioral analysis techniques, sensor data analysis techniques, and so on.

To achieve the benefits described above, the service initially operates in a passive listening state/mode. While in this mode, the service passively listens to utterances made by a patient who may be meeting with or otherwise conversing with a medical practitioner. The service, while passively listening to the utterances, transcribes the utterances into text, resulting in the generation of transcribed utterances. The service temporarily stores these transcribed utterances in a buffer for a first time period, at the expiration of which the service automatically expunges the transcribed utterances from the buffer. Thus, the service avoids retaining patient data for longer than necessary. Typically, this first time period is relatively short, such as about 60 seconds or less.

Prior to a particular transcribed utterance being expunged from the buffer, the service detects a keyword included within the particular transcribed utterance or, alternatively detects a topic of the particular transcribed utterance. Both the keyword and the topic are related to a specific health measure of the patient. For example, it might be the case that the patient is recovering from a surgery, which is one type of health measure. Alternatively, it might be the case that the patient has an ailment and is considering his/her treatment options. The health measure is one that is determined to be relevant to a PROM that is in need of completion. For instance, this specific PROM may be querying about the patient's specific health measure.

In response to detecting the keyword or the topic, the service transitions from operating in the passive listening mode to operating in an active listening mode. This transition starts a second time period during which the service refrains from expunging the buffer (so additional context and information can be obtained and used by the service). The transcribed utterance remains stored in the buffer during the second time period. Typically, the second time period is an unbounded time period that may last until such time as the conversation topic shifts away (or ends) from the patient's specific health measure.

While the service is operating in the active listening state, the service uses natural language processing (NLP) to apply a semantic meaning to a set of transcribed utterances stored in the buffer during the second time period. Notably, the set of transcribed utterances include the earlier transcribed utterance (e.g., the one that triggered the mode shift) and one or more subsequently obtained transcribed utterances.

The service applies a quantitative value, based on the semantic meaning, to the set of transcribed utterances. As a consequence, the service transforms the set of transcribed utterances from being unstructured data to being structured data. As a quick example, suppose the patient said the following, “My knee is in so much pain right now.” The semantic meaning of this utterance relates to knee pain. The service can apply a quantitative value to that utterance as well. For instance, using a rating scale from 0 to 10, with 10 being severe pain, the service may apply a quantitative value of 7.7 to the patient's utterance. This quantitative value is now “structured” data, whereas the phrase “My knee is in so much pain right now” is unstructured data.

Advantageously, the structured data relates to the health measure, and a format of the structured data is designed to correspond to a format of a PROM, which is a report detailing the health measure of the patient. The service uses the structured data to automatically populate one or more fields of the PROM. For instance, if one of the questions of the PROM asks about knee pain, the service can provide the 7.7 answer to that question. By performing these transformative operations, the embodiments are able to greatly assist medical practitioners in populating a PROM and in satisfying various reporting requirements.

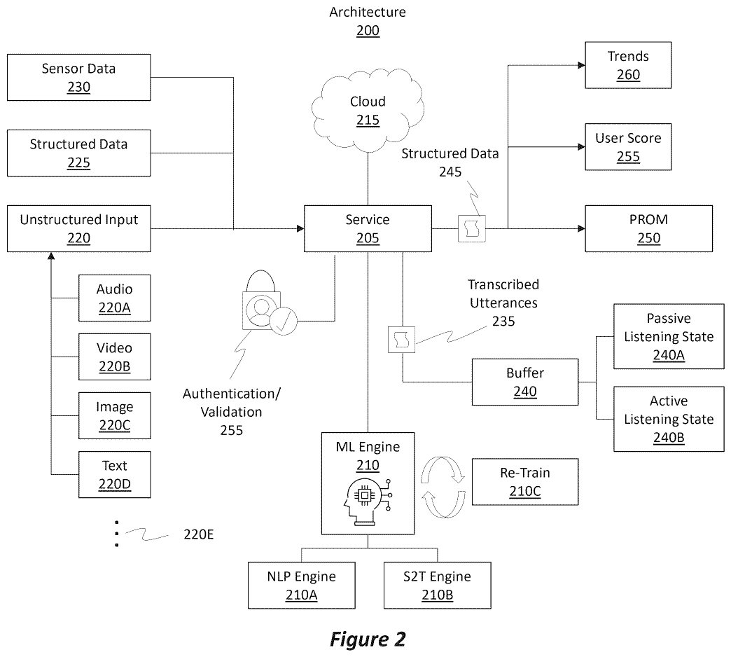

Having just described some of the various benefits, advantages, and practical applications of the disclosed embodiments, attention will now be directed to FIG. 2. FIG. 2 shows an example computing architecture 200 that includes a service 205.

As used herein, the term “service” refers to an automated program that is tasked with performing different actions based on input. In some cases, service 205 can be a deterministic service that operates fully given a set of inputs and without a randomization factor. In other cases, service 205 can be or can include a machine learning (ML) or artificial intelligence engine, such as ML engine 210. The ML engine 210 enables the service 205 to operate even when faced with a randomization factor.

As used herein, reference to any type of machine learning or artificial intelligence may include any type of machine learning algorithm or device, convolutional neural network(s), multilayer neural network(s), recursive neural network(s), deep neural network(s), decision tree model(s) (e.g., decision trees, random forests, and gradient boosted trees) linear regression model(s), logistic regression model(s), support vector machine(s) (“SVM”), artificial intelligence device(s), or any other type of intelligent computing system. Any amount of training data may be used (and perhaps later refined) to train the machine learning algorithm to dynamically perform the disclosed operations.

ML engine 210 may include a natural language processing (NLP) engine 210A. ML engine 210 may also include a speech-to-text (S2T) engine 210B. Also, as will be described in more detail later, the ML engine 210 can be subjected to an initial training phase and then later subjected to a re-training phase (e.g., re-train 210C) or a fine-tuning phase in order to improve its learning abilities.

In some implementations, service 205 is a cloud service operating in a cloud 215 environment. In some implementations, service 205 is a local service operating on a local device. In some implementations, service 205 is a hybrid service that includes a cloud component operating in the cloud 215 and a local component operating on a local device. These two components can communicate with one another.

Generally, service 205 is tasked with accessing input data and using that input data to automatically populate a PROM. By way of example, service 205 may generate, receive, or otherwise access unstructured input 220, which may include any type of audio 220A data, video 220B data, image 220C data, or text 220D. The ellipsis 220E demonstrates how other data can also be obtained. Service 205 can also generate, receive, or otherwise access structured data 225 and/or sensor data 230.

As used herein, “structured” data refers to data that is stored in a predefined format while “unstructured’ data can be a conglomeration of varied data types that are stored together in their native formats and refers to data that does not follow a predefined format. That is, unstructured data lacks a consistent schema or data model.

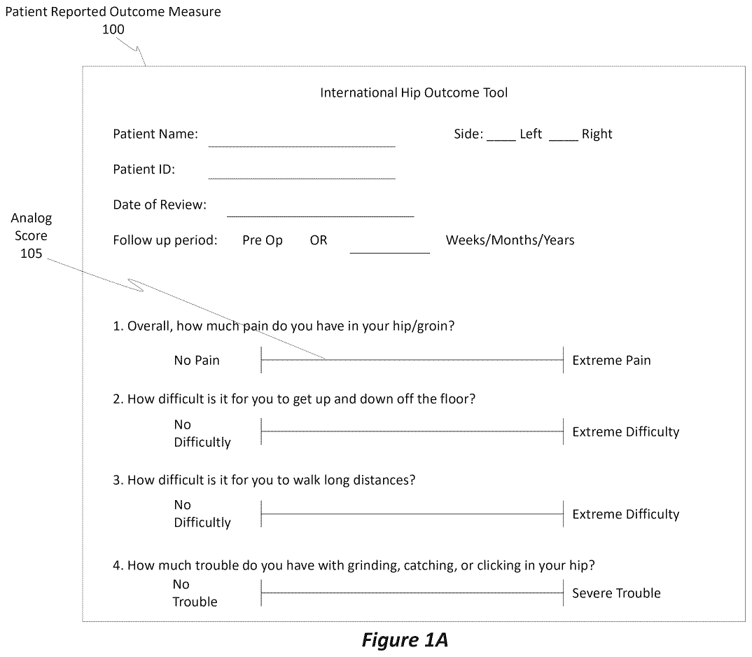

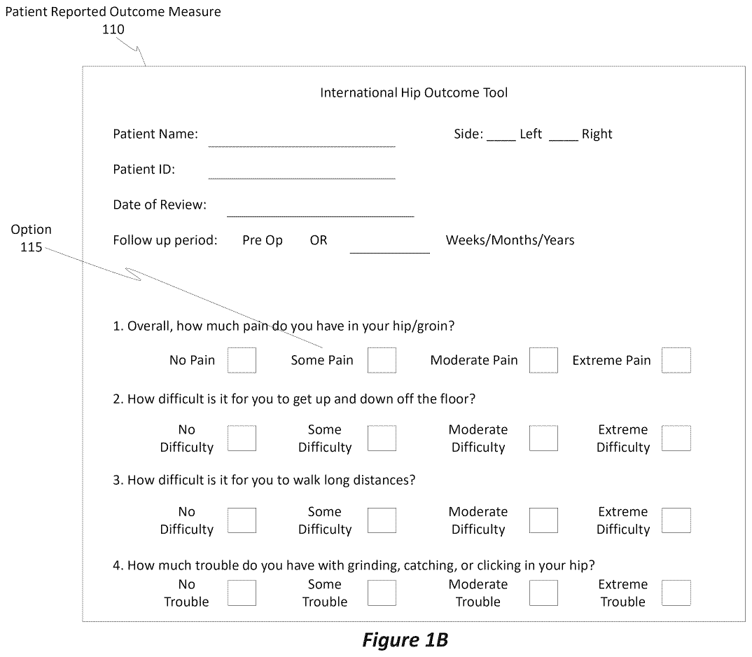

An example of structured data would be a specific 1-5 star rating for a product. Another example of structured data would be a specific score (e.g., 6.5) on the pain rating shown in FIG. 1 by the analog score 105. Yet another example of structured data would be an answer to a checkbox question listing multiple optional answers, such as “No Pain,” “Slight Pain,” “Medium Pain,” and “High Pain.” Selection of any one of these options would result in the generation of structured data.

An example of unstructured data would be a user's typewritten comment on the quality of the product. For instance, the language “this product is amazing” is one example of unstructured data. The language “this product is terrible” is another example of unstructured data. The language “my knee hurts so badly” is another example of unstructured data. Thus, a pain score of 8.7 is an example of structured data while the statement “I am in so much pain” is an example of unstructured data. As will be described in more detail shortly, service 205 is configured to transform or otherwise convert unstructured data into structured data.

Regarding the structured data 225, service 205 might receive some structured data as well as some unstructured data. Service 205 might also receive the sensor data 230. Thus, service 205 can operate using any one or combination of unstructured input 220, structured data 225, and sensor data 230.

Regarding at least the unstructured input 220, service 205 is able to transform the unstructured input 220 into structured data. Using the audio 220A unstructured input (e.g., a recorded conversation between a patient and a medical practitioner) as one example, service 205 accesses the audio recording and uses the S2T engine 210B to generate transcribed utterances 235 of the audio 220A. That is, the transcribed utterances 235 is a transcription of the audio recording. These transcribed utterances 235 may be temporarily stored in a buffer 240.

Service 205 can operate in multiple different modes or states, such as a passive listening state 240A (or a passive observation state) or an active listening state 240B (or an active observation state). When service 205 is operating in the passive listening state 240A, service 205 temporarily stores the transcribed utterances 235 (or, more generally, transcribed output) in the buffer 240 for a limited, predefined period of time prior to expunging the transcribed utterances 235 from the buffer 240. This limited, predefined period of time is typically less than about 60 seconds. In some rare circumstances, the limited, predefined period of time might extend up to about 300 seconds.

When a transcribed utterance is generated, service 205 can append or include metadata details for the transcribed utterance, including a timestamp as to when the transcribed utterance was generated. This same timestamp can also generally reflect the time when the transcribed utterance was first stored in the buffer 240. Thus, for each transcribed utterance, a corresponding timestamp can be generated, and service 205 can determine when a given one or more transcribed utterances are to be expunged from the buffer 240 based on those timestamps.

In some implementations, service 205 implements a bundle expungement process in which the trigger for the bundle expungement is based on the one transcribed utterance having the oldest timestamp in the buffer 240. Once the limited, predefined time period elapses with respect to this oldest timestamp, then all transcribed utterances included in the buffer 240 are expunged together at the same time.

As an example, suppose three transcribed utterances are included in the buffer 240. The first transcribed utterance has a timestamp of 00:08:26; the second transcribed utterance has a timestamp of 00:08:56; and the third transcribed utterance has a timestamp of 00:09:12. Further, suppose the limited, predefined time period is 00:01:00 (or 60 seconds) in duration. In this example implementation, once the timeclock reaches a time of 00:09:26, then all three transcribed utterances will be expunged from the buffer 240.

In another implementation, the bundle expungement is based on a storage threshold. For instance, when the amount of data (e.g., transcribed utterances) stored in the buffer 240 reaches the storage threshold, then the expungement process may be triggered. Thus, in this scenario, the expungement is not based on a time factor but rather is based on a storage amount factor.

In other implementations, service 205 implements a rolling expungement process in which each individual transcribed utterance is expunged from the buffer 240 once that that transcribed utterance has been in the buffer 240 for the limited, predefined time period.

As an example, suppose three transcribed utterances are included in the buffer 240. The first transcribed utterance has a timestamp of 00:08:26; the second transcribed utterance has a timestamp of 00:08:56; and the third transcribed utterance has a timestamp of 00:09:12. Further, suppose the limited, predefined time period is 00:01:00 (or 60 seconds) in duration. In this example implementation, once the timeclock reaches a time of 00:09:26, then only the first transcribed utterance will be expunged from the buffer 240. Once the timeclock reaches a time of 00:09:56, then only the second transcribed utterance will be expunged from the buffer 240. To complete the example, once the timeclock reaches a time of 00:10:12, then only the third transcribed utterance will be expunged from the buffer 240. Thus, different buffer expungement techniques can be implemented.

While service 205 is operating in the passive listening state 240A, service 205 is analyzing the transcribed utterances 235 stored in the buffer 240 to detect either a keyword included in the transcription or a detected topic embodied by the transcription. The detected topic can be determined using the NLP engine 210A. As an example, suppose the transcribed utterances 235 included the following transcribed text: “I am currently experiencing a lot of pain.” “The pain is in my right knee.” “I am still recovering from my knee surgery.” The “topic” of the combination of these three transcribed utterances relates to “knee pain after surgery.”

Service 205 is tasked with attempting to assist in the completion of a PROM. To do so, service 205 can passively listen to a patient's conversation with a medical practitioner. If the conversation is not related to the questions in the PROM, then service 205 remains in the passive listening state 240A. On the other hand, if the conversation shifts and begins to focus on questions or content included in the PROM, then service 205 will transition from operating in the passive listening state 240A to operating in the active listening state 240B. This transition is based on the detection of certain keywords or detected topics identified within the transcribed utterances 235 with respect to the questions of the PROM. That is, the keywords and topics can be obtained from analyzing the PROM, and the service 205 can detect which keywords or topics are relevant to answering the questions recited in the PROM.

Using the patient reported outcome measure 100 of FIG. 1 as an example, the first question asks the following: “Overall, how much pain do you have in your hip/groin?” The topic for this question is hip/groin pain. If service 205 determines that the patient and the medical practitioner are discussing this topic, then service 205 can transition from the passive listening mode to the active listening mode. Similarly, if service 205 detects certain keywords (e.g., perhaps “pain,” “hip,” or “groin”) in the transcribed utterances, then service 205 can transition states or modes. On the other hand, if service 205 determines that the conversation is focused on a topic other than “pain” in the “hip” or “groin” area, then the service 205 may remain in the passive listening mode. Having the service 205 operating in the different states is beneficial to help protect the patient's privacy.

When service 205 transitions to the active listening state 240B, a second time period starts. During this second time period, service 205 refrains from expunging the buffer 240 and instead attempts to build up a log of the conversation between the patient and the medical practitioner. This log is used in an attempt to generate structured data 245, which will be used to automatically populate the PROM 250.

For instance, while service 205 is operating in the active listening state 240B, service 205 uses the NLP engine 210A to determine or apply a semantic meaning to the set of transcribed utterances that are being stored in the buffer 240 during the second time period. Typically, this set of transcribed utterances will include the transcribed utterances that triggered the state transition or switch for the service 205 as well as one or more subsequently obtained transcribed utterances.

Service 205 then applies a quantitative value, based on the semantic meaning, to the set of transcribed utterances. As a result, service 205 transforms the set of transcribed utterances stored in the buffer 240 from being unstructured data to being structured data 245.

Notably, the structured data 245 typically relates to a health measure of the patient. Also, the format of the structured data 245 is designed to correspond to the format of the PROM 250, which is the report detailing the health measure of the patient. Service 205 then uses the structured data 245 to automatically populate one or more fields of the PROM 250. Service 205 may also generate a user score 255 for the patient and/or trends 260 for the patient. Further details on these aspects will be provided later.

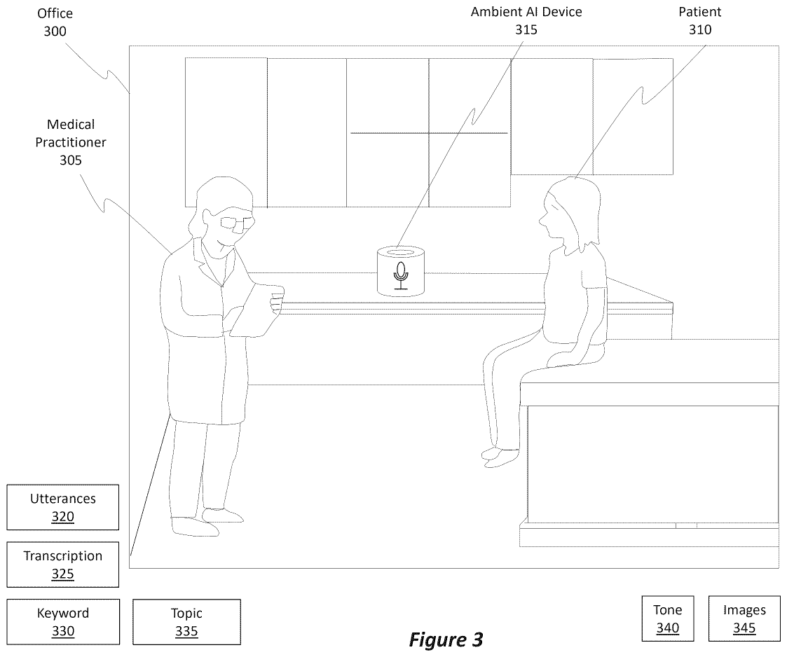

At this point, an example will be helpful. As such, attention will now be directed to FIG. 3.

FIG. 3 shows an example doctor's office 300 in which a medical practitioner 305 is conversing with a patient 310 regarding the patient's health status. Notice, office 300 is shown as including an ambient artificial intelligence (AI) device 315, which is one example implementation of service 205 from FIG. 2.

The ambient AI device 315 is passively listening to the conversation between the medical practitioner 305 and the patient 310. By passively listening, it is meant that the ambient AI device 315 is operating in the passive listening state 240A from FIG. 2 and is expunging the buffer 240 fairly frequently (or rather, at a first expungement rate). Thus, ambient AI device 315 is listening for utterances 320 and is generating a transcription 325 of those utterances 320.

Ambient AI device 315 has knowledge or information relating to the format and content of a PROM for this particular patient 310. Thus, ambient AI device 315 is listening for a keyword 330 or a topic 335 embodied in the transcription 325 relating to the format, content, or questions of the PROM.

In response to detection of the keyword 330 or the topic 335, the ambient AI device 315 will transition to the active listening state and will attempt to determine a semantic meaning for the transcribed utterances as well as a quantitative value for those utterances. In some implementations, the ambient AI device 315 can also determine a tone 340 of the patient's speech in an effort to better predict or determine the semantic meaning and the quantitative value.

For instance, if the tone 340 is indicative that the patient 310 is weeping, then the ambient AI device 315 may further weight its determined quantitative value (e.g., as being more serious). On the other hand, if the tone 340 is indicative that the patient 310 is lethargic, then the ambient AI device 315 may apply a reduced weight to its determined quantitative value (e.g., as being less serious).

Similarly, the ambient AI device 315 can optionally obtain images 345 of the patient 310 to assist in determining the semantic meaning and/or the quantitative value. For instance, the images 345 can be subjected to an image analysis to determine whether visible signs exist with respect to the patient's condition, such as bruising, stiches, lacerations, and so on. If visibly present, then the ambient AI device 315 can also dynamically modify the quantitative value.

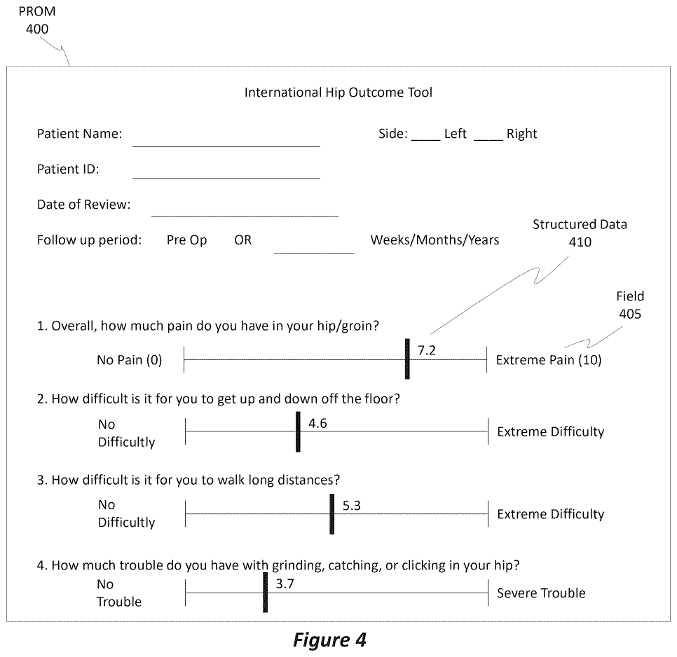

After the ambient AI device 315 has generated the quantitative value, the ambient AI device 315 can automatically populate a PROM using that information, as shown in FIG. 4. To illustrate, FIG. 4 shows a PROM 400 that includes a number of questions along with corresponding fields for a user answer, such as field 405.

Based on listening to the conversation between the medical practitioner 305 and the patient 310, the ambient AI device 315 was able to generate a quantitative value that appropriately answers question #1. That is, the ambient AI device 315 was able to use the generated structured data 410 to automatically populate the field 405. In this example scenario, the structured data 410 indicates a value of 7.2 on the pain scale for question #1. This 7.2 value was based on the content of the conversation between the patient and the medical practitioner.

An example of the conversation between the medical practitioner 305 and the patient 310 may be as follows. Doctor: “So, how are you feeling today?” Patient: “I'm doing ok, but I'm in a little pain.” Doctor: “Oh yeah?” “Where are you hurting?” Patient: “My hip is hurting.” Doctor: “How much is it hurting?” Patient: “Well, when I am sitting, it's ok, but when I'm moving it hurts quite a bit.” “In fact, I have a sharp, shooting pain when I walk.”

During the first statement made by the doctor, the ambient AI device 315 may be operating in the passive listening state because that first statement has little or no relevance to any of the questions in the PROM. In response to the patient saying, “I'm in a little pain,” the ambient AI device 315 may transition to the active listening state because a keyword related to question #1 (or perhaps any of the other questions) deals with pain.

From there, the ambient AI device is actively listening to the conversation. The patient mentions his hip. From that information, the ambient AI device can connect that the pain is likely related to the patient's hip. As a result, the conversation is likely quite relevant to question #1 of the PROM 400.

Subsequently, the patient describes the type of pain and the relative amount of pain. Notice, the patient does not provide a quantitative value for the amount of pain the patient is in. Despite this omission, the ambient AI device is able to predict, gauge, or otherwise determine a relative amount of pain (corresponding to the scale used by the PROM 400) the patient is in. In this example, the determined about of pain (relative to the scale used by the PROM 400) is selected to be a value of 7.2 (out of a maximum value of 10 on the PROM 400). Furthermore, the ambient AI device automatically populated the field 405 using the structured data 410 generated by the ambient AI device. In this case, the structured data 410 is the pain value 7.2.

In the event the conversation between the medical practitioner 305 and the patient 310 of FIG. 3 shifts away from content related to any of the questions of the PROM 400, then the ambient AI device 315 can transition back to the passive listening state. Similarly, the ambient AI device 315 can make that transition in response to other conditions, such as silence for a predefined amount of time (perhaps suggesting the doctor's room is empty or is occupied by a single, silent individual).

The ambient AI device is able to monitor the conversation in an attempt to answer the other questions of the PROM 400 as well. Also, if not all of the questions of the PROM 400 have been answered by the ambient AI device, some embodiments are able to trigger an audio or visual cue to the medical practitioner 305 to prompt or remind the medical practitioner 305 regarding the deficient answers.

By way of example, if the ambient AI device 315 determines that the conversation between the medical practitioner 305 and the patient 310 is wrapping up without resolution of the PROM 400 (or has already wrapped up), then the ambient AI device 315 can play a message from its speaker, such as “A quick reminder doctor, we still need some answers for the PROM.” Additionally, or alternatively, the ambient AI device 315 can light one or more light emitting diodes (LEDS) on its surface to act as a reminder for the medical practitioner 305. In some implementations, the ambient AI device 315 can also trigger a text message, email, or call to the medical practitioner's smart device to remind the medical practitioner that the answers to the PROM 400 are still needed. Thus, multiple different techniques are available to remind or prompt the medical practitioner regarding a deficiency in answering the PROM 400. Such reminders can help ensure that the PROM is fully and adequately completed.

It should be noted how FIG. 3 shows the ambient AI device 315 as a standalone unit. In some implementations, however, service 205 of FIG. 2 can be implemented as a part of a laptop, desktop, tablet, smart phone, wearable device, or any other smart device. Thus, it is not a requirement that service 205 be a standalone unit.

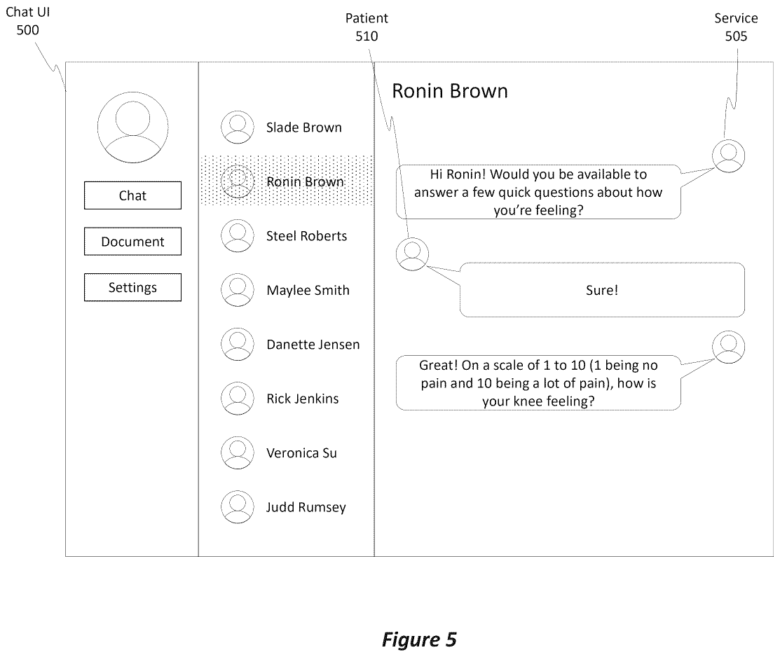

FIG. 5 shows another example use of service 205. In particular, FIG. 5 shows a chat user interface (UI) 500 in which the patient 510 is conversing either with the medical practitioner or perhaps even with the service 505, which is an example implementation of service 205 from FIG. 2. In this scenario, service 505 is tasked with collecting the information needed to complete the PROM 400 of FIG. 4. Here, service 505 is asking the patient 510 a number of questions related to the PROM 400. Service 505 will then apply a semantic meaning and generate a quantitative value for that data. Thus, different techniques are available for acquiring or collecting the information used to complete the PROM 400.

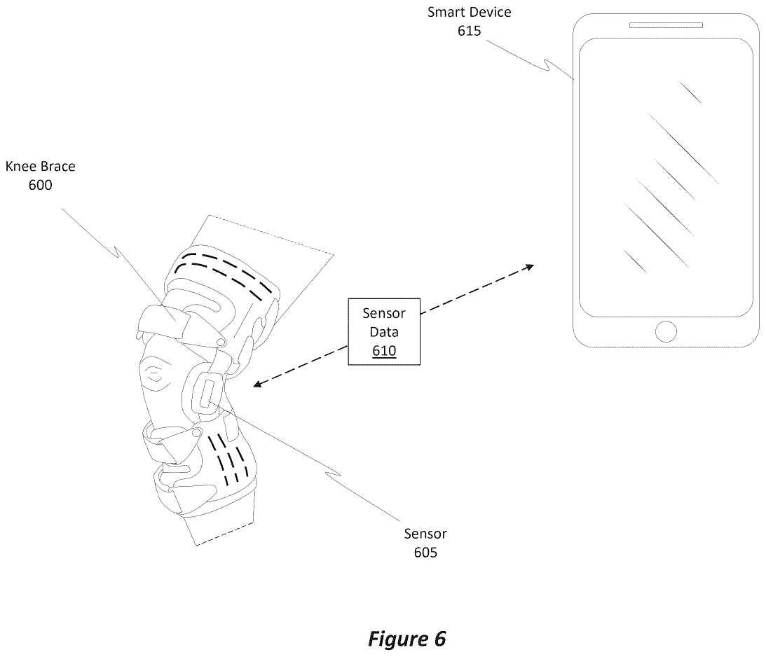

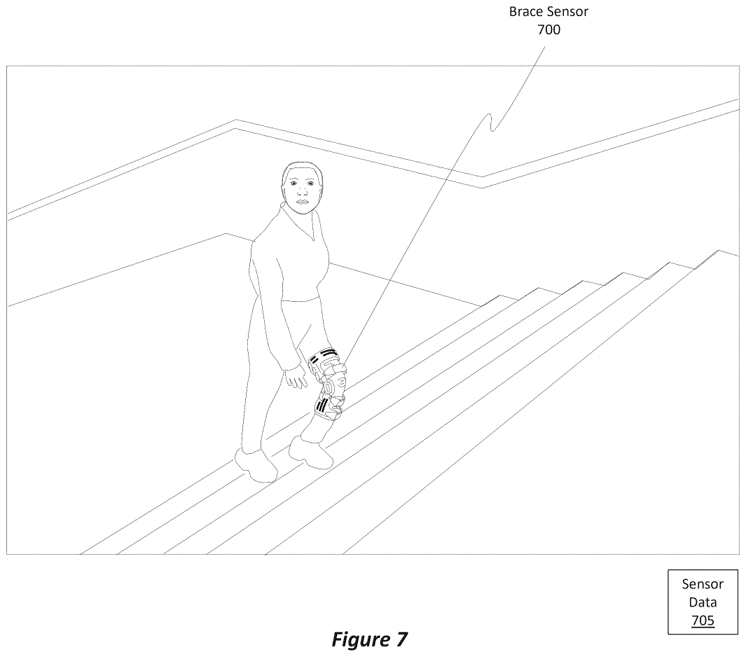

FIGS. 6 and 7 show additional examples of collection techniques. FIG. 6 shows a knee brace 600 that has been equipped with a sensor 605, such as perhaps some type of accelerometer, GPS, inertial measurement unit (IMU), or any other motion sensing device. Sensor 605 is structured to measure the movement of the brace 600 when worn by a patient. Sensor 605 may generate sensor data 610, which can be representative of the sensor data 230 from FIG. 2. Sensor 605 can transmit the sensor data 610 to a user device, such as a smart device 615, which may be implementing the service 205 from FIG. 2. Service 205 may then use the sensor data 610 to determine the quantitative values described herein. FIG. 7 is illustrative.

FIG. 7 shows an example scenario where a patient is wearing a brace that is equipped with a brace sensor 700, similar to sensor 605 of FIG. 6. Here, the patient is walking up stairs. The brace sensor 700 is generating sensor data 705 describing the patient's movements, particularly while the patient is walking up the stairs. Service 205 is able to analyze the sensor data 705 to determine whether walking up the stairs is a labored process for the patient or is an easy process for the patient. For instance, if the sensor data 705 indicates that the patient has to take frequent stops, or the patient's gait is abnormal, or the range of motion of the patient's knee is limited, the service 205 can determine that the patient is likely uncomfortable or in pain. This determination can be used to generate the structured data 245 of FIG. 2.

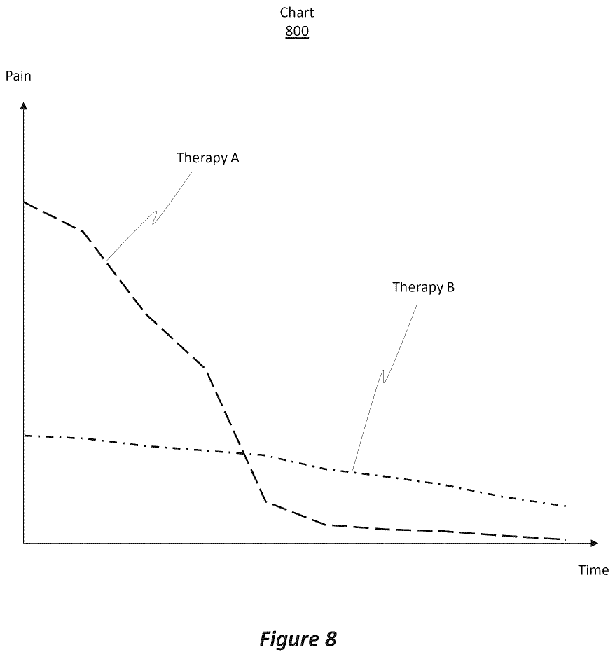

One of the benefits of generating the structured data relates to the ability for patients and medical practitioners to determine the effectiveness of a treatment for a given ailment. FIG. 8 is illustrative.

FIG. 8 shows a chart 800 that plots the various user scores or outcome measurements of patients who have had different treatments for the same ailment. For instance, chart 800 shows therapy A and therapy B. It might be the case that both therapies A and B are for treating a knee injury. As an example, therapy A might be a surgery while therapy B might be physical therapy.

Chart 800 plots the patient's pain (y axis) over a given time period (x axis). In this example scenario, patients who followed therapy A initially experienced a relatively higher amount of pain as compared to patients who followed therapy B. Later, however, those same patients experienced relatively less pain as compared to patients who followed therapy B.

The user score 255 from FIG. 2 can assist in determining plot data, such as the data plotted in chart 800. User scores can reflect the relative degree of success a patient has with a given treatment. The user score can be plotted over time to assist a patient in seeing the progress he/she has made over time. Also, the information can be used to help patients determine which treatment is likely best for them. The user score can reflect the patient's tracked pain (or some other physiological or mental characteristic) over time.

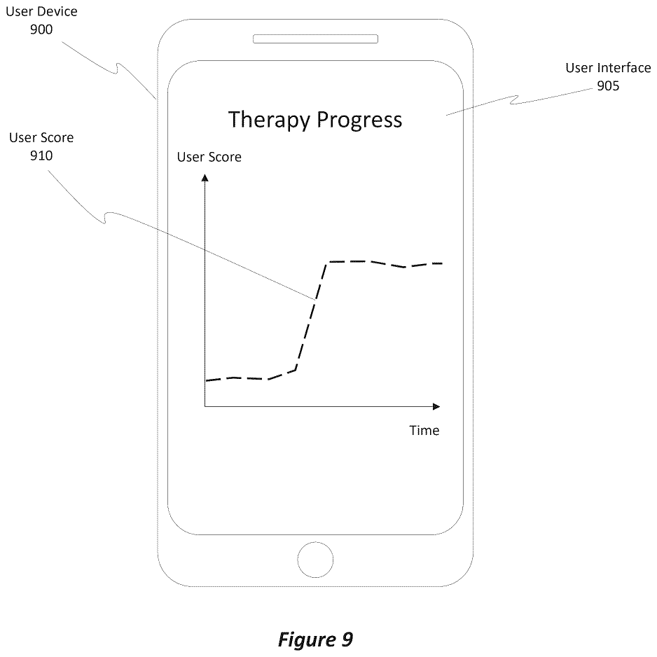

FIG. 9 shows a user device 900 that is displaying a user interface 905. User interface 905 is currently plotting the user's score (e.g., user score 910) over a time period. User interface 905 is particularly beneficial because it can help patients gauge their progress over time. For instance, a user score can be generated for a user undergoing therapy, and the score can rate a certain metric, such as progress or pain. These scores can be compiled and plotted over time to track the user's progress.

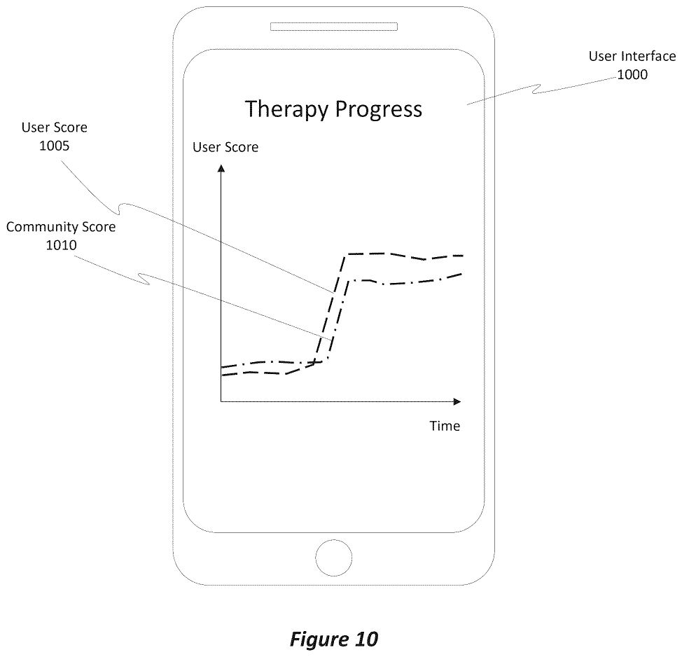

FIG. 10 shows another user interface 1000 plotting a user score 1005. Here, however, user interface 1000 is also plotting the community scores (e.g., community score 1010) for other patients who have followed the same treatment as the patient having the user score 1005. User interface 1000 is thus providing a baseline metric for the patient to gauge how well he/she is progressing relative to other patients with a similar ailment or who went through the same treatment. One beneficial feature of a PROM is that they can be used to establish a baseline against which response to subsequent treatments can be compared. In some scenarios, PROMs are used to describe the patient's perceived level of disability prior to a treatment as well as during the treatment and after the treatment.

In this example scenario, a higher user score indicates a relatively higher success rate for the treatment. Here, this particular user (i.e. the one corresponding to user score 1005) is progressing better than the community average. That is, the community score 1010 may be an aggregation or an average of other, similarly situated patients. For instance, if the patient has a certain sex, age, and/or physical characteristic, the community score 1010 may be for other patients that generally match the same sex, age, and physical characteristic. In other scenarios, the community score 1010 may be a macro score and simply reflect all patients who underwent the same treatment, without regard to granular characteristic differences.

The following discussion now refers to a number of methods and method acts that may be performed. Although the method acts may be discussed in a certain order or illustrated in a flow chart as occurring in a particular order, no particular ordering is required unless specifically stated, or required because an act is dependent on another act being completed prior to the act being performed.

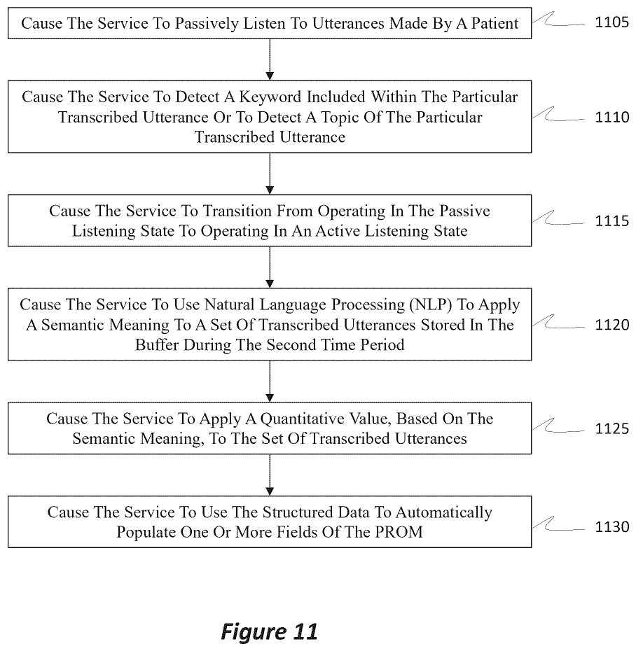

Attention will now be directed to FIG. 11, which illustrates a flowchart of an example method 1100 for using an ambient AI device to collect PROM-related data and for automatically populating the PROM using that collected data. Method 1100 can be performed within the architecture 200 of FIG. 2. Method 1100 can also be performed by service 205.

While a service is operating in a passive listening state (or a passive observation state), act 1105 includes causing the service to passively listen (or passively observe) to utterances (or output) made by a patient. The service, while passively listening (or passively observing) to the utterances (or output), transcribes the utterances (or output) into text resulting in generation of transcribed utterances (or transcribed output). Also, the service temporarily stores the transcribed utterances (or transcribed output) in a buffer for a first time period. At the expiration of that time period, the service automatically expunges the transcribed utterances (or transcribed output) from the buffer. Going forward, reference to “listening” can be replaced with “observation” and references to “utterances” can be replaced with “output.”

Prior to a particular transcribed utterance being expunged from the buffer, act 1110 includes causing the service to detect a keyword included within the particular transcribed utterance or to detect a topic of the particular transcribed utterance. Notably, both the keyword and the topic are related to a specific health measure of the patient.

The specific health measure may relate to any type of health measure. In one example scenario, the specific health measure relates to a surgery (or the results of the surgery, such as pain, range of motion, etc.) the patient previously underwent, and the keyword or the topic also relate to the surgery. The specific health measure can relate to non-surgical treatments as well, such as any form of physical therapy. For example, the specific health measure may relate to a physical therapy the patient previously or is currently undergoing, and the keyword or the topic relate to the physical therapy.

In some implementations, the service, prior to transitioning to the active listening state, triggers an audible prompt to the patient. This audible prompt may include language related to the specific health measure. This audible prompt can operate as a reminder or a trigger for the medical practitioner to be sure to complete the PROM with the patient.

In response to detecting the keyword or the topic, act 1115 includes causing the service to transition from operating in the passive listening state to operating in an active listening state. The service transitioning to the active listening state starts a second time period during which the service refrains from expunging the buffer. The particular transcribed utterance remains stored in the buffer during the second time period. Typically, though not necessarily, the second time period is longer than the first time period. Often, the second time period is unbounded or is not predefined. That is, while the first time period is a predefined time period, the second time period may not be predefined.

While the service is operating in the active listening state, act 1120 includes causing the service to use natural language processing (NLP) to apply a semantic meaning to a set of transcribed utterances stored in the buffer during the second time period. The set of transcribed utterances include the particular transcribed utterance and one or more subsequently obtained transcribed utterances.

Act 1125 includes causing the service to apply a quantitative value, based on the semantic meaning, to the set of transcribed utterances. Consequently, the service transforms the set of transcribed utterances from being unstructured data to being structured data. The structured data relates to the health measure. Also, a format of the structured data is designed to correspond to a format of a patient reported outcome measure (PROM), which is a report detailing the health measure of the patient. In one example, the format of the PROM may include a question that is to be answered using a numeric answer. Consequently, the quantitative value will operate as the numeric answer.

Often, the quantitative value is a ranked response to a question included in the PROM. For instance, the quantitative value may be a numeric value within a range of values (e.g., from 0 to 10) representing a certain condition, such as perhaps comfort or pain. As another example, the quantitative value may be a selection of a checkbox option that is available in the PROM.

Act 1130 includes causing the service to use the structured data to automatically populate one or more fields of the PROM. In some scenarios, the PROM, after being automatically populated using the structured data, is uploaded to a database or an electronic medical record (EMR). Optionally, the database may be a first-party database or it may be a third-party database, such as perhaps a CMS database. In some scenarios, the one or more fields of the PROM are answers to questions having topics related to the health measure of the patient.

In some embodiments, the quantitative value is stored in a profile for the patient. The profile can maintain a log of quantitative values for the patient over time, and the service can chart the log of quantitative values. The service can also collect community quantitative values for other patients whose same health measure was tracked. Similarly, the service can plot the community quantitative values and the patient's quantitative value in a chart. The service may further collect sensor data from a sensor worn by the patient, and the quantitative value can also be based on the sensor data.

In some scenarios, the service may transition back to the passive listening state. This transition back to the passive listening state may occur in response to a detected change in topic in the patient's utterances. In some scenarios, the service is caused to transition back to the passive listening state in response to a determination that no utterances are being made by the patient for a threshold amount of time.

In some implementations, the format of the PROM is a checkbox format. In such scenarios, the format of the structured data corresponds to options that are available in the checkbox format. In some implementations, the format of the PROM is a numerical format. In such scenarios, the format of the structured data corresponds to numeric values that are available in the numerical format.

Optionally, after the PROM is automatically populated, the disclosed service can submit the PROM to the patient. The service can also receive validation input from the patient, where the validation input validates the PROM.

Returning briefly to FIG. 2, it was previously mentioned how the ML engine 210 can be re-trained, as shown by re-train 210C. In some implementations, the ML engine 210 is initially subjected to a first training phase during which the ML engine 210 is trained generally on PROM question and answer data as well as patient response data. During a second training phase, which is the re-training phase, the ML engine 210 can be further tuned or trained based on updated PROM data or changes to PROMs as well as updated language corresponding to the PROM questions and answers. Thus, the ML engine 210 can be subjected to multiple different phases of training so as to improve its learning abilities.

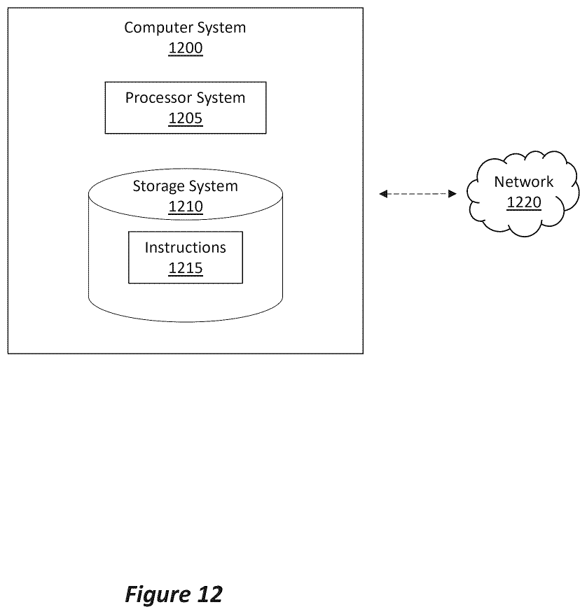

Attention will now be directed to FIG. 12 which illustrates an example computer system 1200 that may include and/or be used to perform any of the operations described herein. For example, computer system 1200 can implement service 205 of FIG. 2.

Computer system 1200 may take various different forms. For example, computer system 1200 may be embodied as a tablet, a desktop, a laptop, a mobile device, or a standalone device, such as those described throughout this disclosure. Computer system 1200 may also be a distributed system that includes one or more connected computing components/devices that are in communication with computer system 1200.

In its most basic configuration, computer system 1200 includes various different components. FIG. 12 shows that computer system 1200 includes a processor system 1205 comprising one or more processor(s) (aka a “hardware processing unit”) and a storage system 1210.

Regarding the processor(s) of the processor system 1205, it will be appreciated that the functionality described herein can be performed, at least in part, by one or more hardware logic components. For example, and without limitation, illustrative types of hardware logic components/processors that can be used include Field-Programmable Gate Arrays (“FPGA”), Program-Specific or Application-Specific Integrated Circuits (“ASIC”), Program-Specific Standard Products (“ASSP”), System-On-A-Chip Systems (“SOC”), Complex Programmable Logic Devices (“CPLD”), Central Processing Units (“CPU”), Graphical Processing Units (“GPU”), or any other type of programmable hardware.

As used herein, the terms “executable module,” “executable component,” “component,” “module,” “service,” or “engine” can refer to hardware processing units or to software objects, routines, or methods that may be executed on computer system 1200. The different components, modules, engines, and services described herein may be implemented as objects or processors that execute on computer system 1200 (e.g. as separate threads).

Storage system 1210 may be physical system memory, which may be volatile, non-volatile, or some combination of the two. The term “memory” may also be used herein to refer to non-volatile mass storage such as physical storage media. If computer system 1200 is distributed, the processing, memory, and/or storage capability may be distributed as well.

Storage system 1210 is shown as including executable instructions 1215. The executable instructions 1215 represent instructions that are executable by the processor system 1205 to perform the disclosed operations, such as those described in the various methods.

The disclosed embodiments may comprise or utilize a special-purpose or general-purpose computer including computer hardware, such as, for example, one or more processors and system memory, as discussed in greater detail below. Embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are “physical computer storage media” or a “hardware storage device.” Furthermore, computer-readable storage media, which includes physical computer storage media and hardware storage devices, exclude signals, carrier waves, and propagating signals. On the other hand, computer-readable media that carry computer-executable instructions are “transmission media” and include signals, carrier waves, and propagating signals. Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in the form of computer-executable instructions, data, or data structures and that can be accessed by a general-purpose or special-purpose computer.

Computer system 1200 may also be connected (via a wired or wireless connection) to external sensors (e.g., one or more remote cameras) or devices via a network 1220. For example, computer system 1200 can communicate with any number devices or cloud services to obtain or process data. In some cases, network 1220 may itself be a cloud network. Furthermore, computer system 1200 may also be connected through one or more wired or wireless networks to remote/separate computer systems(s) that are configured to perform any of the processing described with regard to computer system 1200.

A “network,” like network 1220, is defined as one or more data links and/or data switches that enable the transport of electronic data between computer systems, modules, and/or other electronic devices. When information is transferred, or provided, over a network (either hardwired, wireless, or a combination of hardwired and wireless) to a computer, the computer properly views the connection as a transmission medium. Computer system 1200 will include one or more communication channels that are used to communicate with the network 1220. Transmissions media include a network that can be used to carry data or desired program code means in the form of computer-executable instructions or in the form of data structures. Further, these computer-executable instructions can be accessed by a general-purpose or special-purpose computer. Combinations of the above should also be included within the scope of computer-readable media.

Upon reaching various computer system components, program code means in the form of computer-executable instructions or data structures can be transferred automatically from transmission media to computer storage media (or vice versa). For example, computer-executable instructions or data structures received over a network or data link can be buffered in RAM within a network interface module (e.g., a network interface card or “NIC”) and then eventually transferred to computer system RAM and/or to less volatile computer storage media at a computer system. Thus, it should be understood that computer storage media can be included in computer system components that also (or even primarily) utilize transmission media.

Computer-executable (or computer-interpretable) instructions comprise, for example, instructions that cause a general-purpose computer, special-purpose computer, or special-purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the embodiments may be practiced in network computing environments with many types of computer system configurations, including personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, and the like. The embodiments may also be practiced in distributed system environments where local and remote computer systems that are linked (either by hardwired data links, wireless data links, or by a combination of hardwired and wireless data links) through a network each perform tasks (e.g. cloud computing, cloud services and the like). In a distributed system environment, program modules may be located in both local and remote memory storage devices.

The disclosed embodiments can be implemented in numerous different ways, as described in the various different clauses recited below.

Clause 1. A computer system comprising: a processor system; and a storage system that stores instructions that are executable by the processor system to cause the computer system to: while a service is operating in a passive observation state, cause the service to passively observe output made by a patient, wherein the service, while passively observing the output made by the patient, transcribes the output into text resulting in generation of transcribed output, and wherein the service temporarily stores the transcribed output in a buffer for a first time period at the expiration of which the service automatically expunges the transcribed output from the buffer; prior to a particular transcribed output being expunged from the buffer, cause the service to detect a keyword included within the particular transcribed output or to detect a topic of the particular transcribed output, wherein both the keyword and the topic are related to a specific health measure of the patient; in response to detecting the keyword or the topic, cause the service to transition from operating in the passive observation state to operating in an active observation state, wherein the service transitioning to the active observation state starts a second time period during which the service refrains from expunging the buffer, and wherein the particular transcribed output remains stored in the buffer during the second time period; while the service is operating in the active observation state, cause the service to use natural language processing (NLP) to apply a semantic meaning to a set of transcribed output stored in the buffer during the second time period, the set of transcribed output comprising the particular transcribed output and one or more subsequently obtained transcribed output; cause the service to apply a quantitative value, based on the semantic meaning, to the set of transcribed output, such that the service transforms the set of transcribed output from being unstructured data to being structured data, wherein the structured data relates to the health measure, and wherein a format of the structured data is designed to correspond to a format of a patient reported outcome measure (PROM), which is a report detailing the health measure of the patient; and cause the service to use the structured data to automatically populate one or more fields of the PROM.

Clause 2. The computer system of any preceding clause, wherein the service, prior to transitioning to the active observation state, triggers an audible prompt to the patient, the audible prompt including language related to the specific health measure.

Clause 3. The computer system of any preceding clause, wherein the PROM, after being automatically populated using the structured data, is uploaded to a database.

Clause 4. The computer system of any preceding clause, wherein the specific health measure relates to a surgery the patient previously underwent, and wherein the keyword or the topic relate to the surgery.

Clause 5. The computer system of any preceding clause, wherein the specific health measure relates to a physical therapy the patient previously or is currently undergoing, and wherein the keyword or the topic relate to the physical therapy.

Clause 6. The computer system of any preceding clause, wherein the quantitative value is a ranked response to a question included in the PROM.

Clause 7. The computer system of any preceding clause, wherein the format of the PROM includes a question that is to be answered using a numeric answer, and wherein the quantitative value operates as the numeric answer.

Clause 8. The computer system of any preceding clause, wherein the quantitative value is stored in a profile for the patient, wherein the profile maintains a log of quantitative values for the patient over time, and wherein the service charts the log of quantitative values.

Clause 9. The computer system of any preceding clause, wherein the service collects community quantitative values for other patients whose same health measure was tracked, and wherein the service plots the community quantitative values and the patient's quantitative value in a chart.

Clause 10. The computer system of any preceding clause, wherein the service further collects sensor data from a sensor worn by the patient, and wherein the quantitative value is also based on the sensor data.

Clause 11. A method comprising: while a service is operating in a passive listening state, causing the service to passively listen to utterances made by a patient, wherein the service, while passively listening to the utterances, transcribes the utterances into text resulting in generation of transcribed utterances, and wherein the service temporarily stores the transcribed utterances in a buffer for a first time period at the expiration of which the service automatically expunges the transcribed utterances from the buffer; prior to a particular transcribed utterance being expunged from the buffer, causing the service to detect a keyword included within the particular transcribed utterance or to detect a topic of the particular transcribed utterance, wherein both the keyword and the topic are related to a specific health measure of the patient; in response to detecting the keyword or the topic, causing the service to transition from operating in the passive listening state to operating in an active listening state, wherein the service transitioning to the active listening state starts a second time period during which the service refrains from expunging the buffer, and wherein the particular transcribed utterance remains stored in the buffer during the second time period; while the service is operating in the active listening state, causing the service to use natural language processing (NLP) to apply a semantic meaning to a set of transcribed utterances stored in the buffer during the second time period, the set of transcribed utterances comprising the particular transcribed utterance and one or more subsequently obtained transcribed utterances; causing the service to apply a quantitative value, based on the semantic meaning, to the set of transcribed utterances, such that the service transforms the set of transcribed utterances from being unstructured data to being structured data, wherein the structured data relates to the health measure, and wherein a format of the structured data is designed to correspond to a format of a patient reported outcome measure (PROM), which is a report detailing the health measure of the patient; and causing the service to use the structured data to automatically populate one or more fields of the PROM.

Clause 12. The method of any preceding clause, wherein the second time period is longer than the first time period.

Clause 13. The method of any preceding clause, wherein the first time period is a predefined time period, and wherein the second time period is not predefined.

Clause 14. The method of any preceding clause, wherein the service is caused to transition back to the passive listening state in response to a detected change in topic in the patient's utterances.

Clause 15. The method of any preceding clause, wherein the service is caused to transition back to the passive listening state in response to a determination that no utterances are being made by the patient for a threshold amount of time.

Clause 16. The method of any preceding clause, wherein the one or more fields of the PROM are answers to questions having topics related to the health measure of the patient.

Clause 17. The method of any preceding clause, wherein the format of the PROM is a checkbox format, and wherein the format of the structured data corresponds to options that are available in the checkbox format.

Clause 18. The method of any preceding clause, wherein the format of the PROM is a numerical format, and wherein the format of the structured data corresponds to numeric values that are available in the numerical format.

Clause 19. The method of any preceding clause, wherein the method further includes: after the PROM is automatically populated, submitting the PROM to the patient; and receiving validation input from the patient, the validation input validating the PROM.

Clause 20. A method that is implemented by a cloud-based service, said method comprising: while the cloud-based service is operating in a passive listening state, causing the cloud-based service to passively listen to utterances made by a patient, wherein the cloud-based service, while passively listening to the utterances, transcribes the utterances into text resulting in generation of transcribed utterances, and wherein the cloud-based service temporarily stores the transcribed utterances in a buffer for a first time period at the expiration of which the cloud-based service automatically expunges the transcribed utterances from the buffer; prior to a particular transcribed utterance being expunged from the buffer, causing the cloud-based service to detect a keyword included within the particular transcribed utterance or to detect a topic of the particular transcribed utterance, wherein both the keyword and the topic are related to a specific health measure of the patient; in response to detecting the keyword or the topic, causing the cloud-based service to transition from operating in the passive listening state to operating in an active listening state, wherein the cloud-based service transitioning to the active listening state starts a second time period during which the cloud-based service refrains from expunging the buffer, and wherein the particular transcribed utterance remains stored in the buffer during the second time period; while the cloud-based service is operating in the active listening state, causing the cloud-based service to use natural language processing (NLP) to apply a semantic meaning to a set of transcribed utterances stored in the buffer during the second time period, the set of transcribed utterances comprising the particular transcribed utterance and one or more subsequently obtained transcribed utterances; causing the cloud-based service to apply a quantitative value, based on the semantic meaning, to the set of transcribed utterances, such that the cloud-based service transforms the set of transcribed utterances from being unstructured data to being structured data, wherein the structured data relates to the health measure, and wherein a format of the structured data is designed to correspond to a format of a patient reported outcome measure (PROM), which is a report detailing the health measure of the patient; and causing the cloud-based service to use the structured data to automatically populate one or more fields of the PROM.

The present invention may be embodied in other specific forms without departing from its characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A computer system comprising:
a processor system; and
a storage system that stores instructions that are executable by the processor system to cause the computer system to:
while a service is operating in a passive observation state, cause the service to passively observe output made by a patient, wherein the service, while passively observing the output made by the patient, transcribes the output into text resulting in generation of transcribed output, and wherein the service temporarily stores the transcribed output in a buffer for a first time period at the expiration of which the service automatically expunges the transcribed output from the buffer;
prior to a particular transcribed output being expunged from the buffer, cause the service to detect a keyword included within the particular transcribed output or to detect a topic of the particular transcribed output, wherein both the keyword and the topic are related to a specific health measure of the patient;
in response to detecting the keyword or the topic, cause the service to transition from operating in the passive observation state to operating in an active observation state, wherein the service transitioning to the active observation state starts a second time period during which the service refrains from expunging the buffer, and wherein the particular transcribed output remains stored in the buffer during the second time period;
while the service is operating in the active observation state, cause the service to use natural language processing (NLP) to apply a semantic meaning to a set of transcribed output stored in the buffer during the second time period, the set of transcribed output comprising the particular transcribed output and one or more subsequently obtained transcribed output;
cause the service to apply a quantitative value, based on the semantic meaning, to the set of transcribed output, such that the service transforms the set of transcribed output from being unstructured data to being structured data, wherein the structured data relates to the health measure, and wherein a format of the structured data is designed to correspond to a format of a patient reported outcome measure (PROM), which is a report detailing the health measure of the patient; and
cause the service to use the structured data to automatically populate one or more fields of the PROM.

2. The computer system of claim 1, wherein the service, prior to transitioning to the active observation state, triggers an audible prompt to the patient, the audible prompt including language related to the specific health measure.

3. The computer system of claim 1, wherein the PROM, after being automatically populated using the structured data, is uploaded to a database.

4. The computer system of claim 1, wherein the specific health measure relates to a surgery the patient previously underwent, and wherein the keyword or the topic relate to the surgery.

5. The computer system of claim 1, wherein the specific health measure relates to a physical therapy the patient previously or is currently undergoing, and wherein the keyword or the topic relate to the physical therapy.

6. The computer system of claim 1, wherein the quantitative value is a ranked response to a question included in the PROM.

7. The computer system of claim 1, wherein the format of the PROM includes a question that is to be answered using a numeric answer, and wherein the quantitative value operates as the numeric answer.

8. The computer system of claim 1, wherein the quantitative value is stored in a profile for the patient, wherein the profile maintains a log of quantitative values for the patient over time, and wherein the service charts the log of quantitative values.

9. The computer system of claim 8, wherein the service collects community quantitative values for other patients whose same health measure was tracked, and wherein the service plots the community quantitative values and the patient's quantitative value in a chart.

10. The computer system of claim 1, wherein the service further collects sensor data from a sensor worn by the patient, and wherein the quantitative value is also based on the sensor data.

11. A method comprising:
while a service is operating in a passive listening state, causing the service to passively listen to utterances made by a patient, wherein the service, while passively listening to the utterances, transcribes the utterances into text resulting in generation of transcribed utterances, and wherein the service temporarily stores the transcribed utterances in a buffer for a first time period at the expiration of which the service automatically expunges the transcribed utterances from the buffer;
prior to a particular transcribed utterance being expunged from the buffer, causing the service to detect a keyword included within the particular transcribed utterance or to detect a topic of the particular transcribed utterance, wherein both the keyword and the topic are related to a specific health measure of the patient;
in response to detecting the keyword or the topic, causing the service to transition from operating in the passive listening state to operating in an active listening state, wherein the service transitioning to the active listening state starts a second time period during which the service refrains from expunging the buffer, and wherein the particular transcribed utterance remains stored in the buffer during the second time period;
while the service is operating in the active listening state, causing the service to use natural language processing (NLP) to apply a semantic meaning to a set of transcribed utterances stored in the buffer during the second time period, the set of transcribed utterances comprising the particular transcribed utterance and one or more subsequently obtained transcribed utterances;
causing the service to apply a quantitative value, based on the semantic meaning, to the set of transcribed utterances, such that the service transforms the set of transcribed utterances from being unstructured data to being structured data, wherein the structured data relates to the health measure, and wherein a format of the structured data is designed to correspond to a format of a patient reported outcome measure (PROM), which is a report detailing the health measure of the patient; and
causing the service to use the structured data to automatically populate one or more fields of the PROM.

12. The method of claim 11, wherein the second time period is longer than the first time period.

13. The method of claim 11, wherein the first time period is a predefined time period, and wherein the second time period is not predefined.

14. The method of claim 11, wherein the service is caused to transition back to the passive listening state in response to a detected change in topic in the patient's utterances.

15. The method of claim 11, wherein the service is caused to transition back to the passive listening state in response to a determination that no utterances are being made by the patient for a threshold amount of time.

16. The method of claim 11, wherein the one or more fields of the PROM are answers to questions having topics related to the health measure of the patient.

17. The method of claim 11, wherein the format of the PROM is a checkbox format, and wherein the format of the structured data corresponds to options that are available in the checkbox format.

18. The method of claim 11, wherein the format of the PROM is a numerical format, and wherein the format of the structured data corresponds to numeric values that are available in the numerical format.

19. The method of claim 11, wherein the method further includes:
after the PROM is automatically populated, submitting the PROM to the patient; and
receiving validation input from the patient, the validation input validating the PROM.

20. A method that is implemented by a cloud-based service, said method comprising:
while the cloud-based service is operating in a passive listening state, causing the cloud-based service to passively listen to utterances made by a patient, wherein the cloud-based service, while passively listening to the utterances, transcribes the utterances into text resulting in generation of transcribed utterances, and wherein the cloud-based service temporarily stores the transcribed utterances in a buffer for a first time period at the expiration of which the cloud-based service automatically expunges the transcribed utterances from the buffer;
prior to a particular transcribed utterance being expunged from the buffer, causing the cloud-based service to detect a keyword included within the particular transcribed utterance or to detect a topic of the particular transcribed utterance, wherein both the keyword and the topic are related to a specific health measure of the patient;
in response to detecting the keyword or the topic, causing the cloud-based service to transition from operating in the passive listening state to operating in an active listening state, wherein the cloud-based service transitioning to the active listening state starts a second time period during which the cloud-based service refrains from expunging the buffer, and wherein the particular transcribed utterance remains stored in the buffer during the second time period;
while the cloud-based service is operating in the active listening state, causing the cloud-based service to use natural language processing (NLP) to apply a semantic meaning to a set of transcribed utterances stored in the buffer during the second time period, the set of transcribed utterances comprising the particular transcribed utterance and one or more subsequently obtained transcribed utterances;
causing the cloud-based service to apply a quantitative value, based on the semantic meaning, to the set of transcribed utterances, such that the cloud-based service transforms the set of transcribed utterances from being unstructured data to being structured data, wherein the structured data relates to the health measure, and wherein a format of the structured data is designed to correspond to a format of a patient reported outcome measure (PROM), which is a report detailing the health measure of the patient; and
causing the cloud-based service to use the structured data to automatically populate one or more fields of the PROM.
